(19) United States
(12) Patent Application Publication
(10) Pub. No.: US 2009/0069811 A1
(43) Pub. Date: Mar. 12, 2009

(54) Device and method for fixation of equipment to the head of a patient during neurological diagnosis, therapy or surgery

(75) Inventors: Magnus Lindfors, Stockholm (SE); Thomas Arn, Lidingo (SE); Jouni Frant, Ekero (SE); Jan-Erik Olsson, Linkoping (SE)

Correspondence Address: BUCHANAN, INGERSOLL & ROONEY PC, POST OFFICE BOX 1404, ALEXANDRIA, VA, 22313-1404, US

(73) Assignee: ELEKTA AB (PUBL), Stockholm (SE)

(21) Appl. No.: 11/918,747

(22) PCT Filed: Apr. 26, 2006
(86) PCT No.: PCT/SE2006/000482

§ 371 Date: Oct. 18, 2007

(30) Foreign Application Priority Data
Apr. 29, 2005 (SE) 0501001-2

(51) Int. Cl. A61B 18/18

(52) U.S. Cl. 606/56; 606/54

(57) ABSTRACT

A device is provided for fixation of equipment to the head of a patient during neurological diagnosis, therapy or surgery, comprising a ring-shaped frame having at least three fixation pins which are attachable against the skull of the patient with a contact force for fixation of the device thereto. At least one connecting means is positioned at a location where the frame has a minimum of displacement due to deformation of the frame when the fixation pins are tightened towards the skull, in order to allow fixation of equipment for diagnosis, therapy or surgery to the frame without they being distorted as a consequence of the deformation of the frame. The invention also relates to a method for fixation of the head of a patient by means of such a device.

The present invention relates to a device for fixation of equipment to the head of a patient during neurological diagnosis, therapy or surgery, comprising a ring-shaped frame having at least three fixation pins, which are attachable against the skull of the patient with a contact force for fixation of the device thereto.

The invention also relates to a method for fixation of equipment to the head of a patient by means of such a device.

### BACKGROUND OF THE INVENTION

A frame of the above kind, known in the art, has normally a generally rectangular or square, ring-shaped configuration, wherein it is slipped over the head of the patient and attached to the head by means of pointed fixation pins, provided in the ends of at least three upright pin support members, which penetrates into the skull by e.g. threading through the pin support members. Normally, the frame is located at the level of the mouth of the patient. However, there also exists frames that have a circular or elliptic form and/or are attached by means of fixation pins which are threaded directly through the frame without any need for intermediate pin support members. In the latter case the frame will be located at the level of the forehead of the patient.

When fixation of a frame to the head of a patient for neurological diagnosis, therapy or surgery, in a way as described above, the frame will be deformed or distorted as a consequence of the rather large forces acting between the head and the frame by means of the fixation pins. The frame is in its turn used as a carrier for other equipment employed during the neurological procedures. Therefore the deformation of the frame affect the precision of the diagnosis, therapy or surgery since the equipment may be displaced due to the deformation of the frame.

Examples of equipment which may be carried by the frame during neurological procedures, are indication boxes of transparent acrylic plastic or the like, having fiducials or reference markings being visible in the imaging method used, which are mounted on the frame during diagnosis, to establish the relative position between the frame and the area to examine or treat in the head. Due to the deformation of the frame, the connection between the indication box and the frame must be performed with a certain play to allow mounting. This decreases the precision of the established position.

Other equipment which normally is used in combination with the frame, is some kind of holding fixture for holding instruments, e.g. a surgical instrument or needle for treatment or taking of samples in areas inside the head. For this purpose the frame usually has a rectangular or square form, such that two opposite, straight frame members may function as guide bars for sliding mounting of the holding fixture in desired locations along the guide bars. However, when clamping a rectangular frame to the head, the frame members functioning as guide bars usually gets their largest deformation in the mid portion, i.e. in the area where the holding fixture is placed when treating or taking of samples in deep located, central areas of the head, which may lead to errors when positioning of the instruments.

Furthermore, the frame is often used as a fixture for steady mounting of the patient's head to different kinds of equipments or apparatuses, such as image producing equipment, for image scanning of the head, or equipment for radiation treatment of chosen areas inside the head. The same problems apply also here, i.e. if the fixation points at the frame are displaced due to deformation of the frame, the diagnosis and/or the treatment may be unprecisely performed.

Moreover, recently it has been proposed to use frames made from synthetic materials, such as plastics, instead of from metals, in order to render them inert in respect of the imaging method used and thereby prevent distortion of the images. Synthetic materials often has a tendency to larger deformations when loaded, which will increase the problems outlined above. Improved quality of the images, also make it possible to see the target areas with increased accuracy, which in turn increases the requirements on the equipment.

### SUMMARY OF THE INVENTION

The object of the invention is to eliminate or reduce the above mentioned problems associated with prior art devices, and provide a device for fixation of equipment to the head of a patient, by which it is possible to perform diagnosis, therapy or surgery with a greater degree of precision than before. At least this object is achieved by a device according to claim 1.

The invention also relates to a method for fixation of equipment to the head of a patient, having essentially the same object as above. At least this object is achieved by a method according to claim 4.

Accordingly, the object is achieved by a device and a method, wherein at least one connecting means is provided at a location where the frame has a minimum of displacement due to deformation of the frame caused by tightening of the fixation pins towards the skull, in order to allow fixation of equipment for diagnosis, therapy or surgery to the frame without having said equipment loosing accuracy or shape or being in any way deformed or distorted as a consequence of the deformation of the frame.

The connecting means may be of any arbitrary, appropriate kind allowing connection of any equipment thereto for diagnosis, therapy or surgery. Thus, in its simplest embodiment the connecting means may have the form of a through hole in the frame. The position of the connecting means is in a preferred embodiment determined through calculations of the deformation, such as by the finite element method (FEM). However, naturally the proper position may be determined by load testing and measuring in practice, having the fixation pins loaded with essentially identical forces.

According to the general inventive idea, the frame comprises at least one connecting means located in a position where the displacement due to deformation is minimal. Through this connecting means any arbitrary equipment, having a matching connecting means, may be connected to the frame. In such a case, the connection between the frame and the equipment must be rotation resistant in order to hold the equipment in place.

At least for heavier equipment, it is however preferred that at least two connecting means, at a distance from each other, are provided in the frame, each located in a position where displacement due to deformation is minimal. With such an embodiment, the equipment may be supported in a much more rigid way and deformations in the equipment itself can be prevented. In this case however, the individual connections between the frame and the equipment should be at least limited rotatable, to avoid formation of stresses and possible deformations in the equipment, due to the deformation of the frame.

In a preferred embodiment of the invention, at least four connecting means are provided in the frame. More precisely are the four connecting means provided in pairs on opposite sides of the frame, preferably on the sides of the frame which, in a mounted state on the patient, will be located on each side of the head. In this way it is possible, among others, to mount two straight guide members in parallel to each other, one on each side of the head, to allow displaceable supporting of the equipment, e.g. a holding fixture for a surgical instrument or needle, on each side of the head.

In a hereinafter described and in the drawings shown embodiment of the invention, the frame has a generally rectangular configuration and is provided with upright pin support members in each corner. Described and shown is also an embodiment where the frame is rounded, having an elliptic form, and the fixation pins are supported directly by the frame without any supplementary pin support members. In the first depicted embodiment, the connecting means are formed as holes in parallel to the plane of the ring-shaped frame. In the second depicted embodiment, the connecting means is however formed as holes perpendicular to the plane of the ring-shaped frame, i.e. as holes in the upper edge of the frame.

In one of the figures, the frame is moreover shown with an equipment in form of an indication box or hood, having reference markings which are visible in an imaging method used. To avoid using that kind of bulky indicating box, it has been proposed to use reference markings or fiducials which are integrated into the frame. To minimize errors, such integrated reference markings should also be located in or near positions which have a minimum of displacement due to deformation of the frame.

### BRIEF DESCRIPTION OF THE DRAWINGS

The invention will hereinafter be described, by way of example, with reference to the accompanying drawings, in which:

FIG. 1 is a perspective view of an embodiment of a frame mounted on the head of a patient;

FIG. 2 is a schematic side elevation of a frame according to FIG. 1 in an unloaded condition;

FIG. 3 is a side elevation according to FIG. 2 in a loaded condition with a guide member mounted;

FIG. 4 is a side elevation according to FIG. 3 with a holding fixture mounted onto the guide members;

FIG. 5 is a view from above of FIG. 4;

FIG. 6 is a side elevation according to FIG. 2 in a loaded condition with an indication box mounted; and

FIG. 7 is a perspective view of a second embodiment of a frame mounted on the head of a patient.

### DESCRIPTION OF PREFERRED EMBODIMENTS

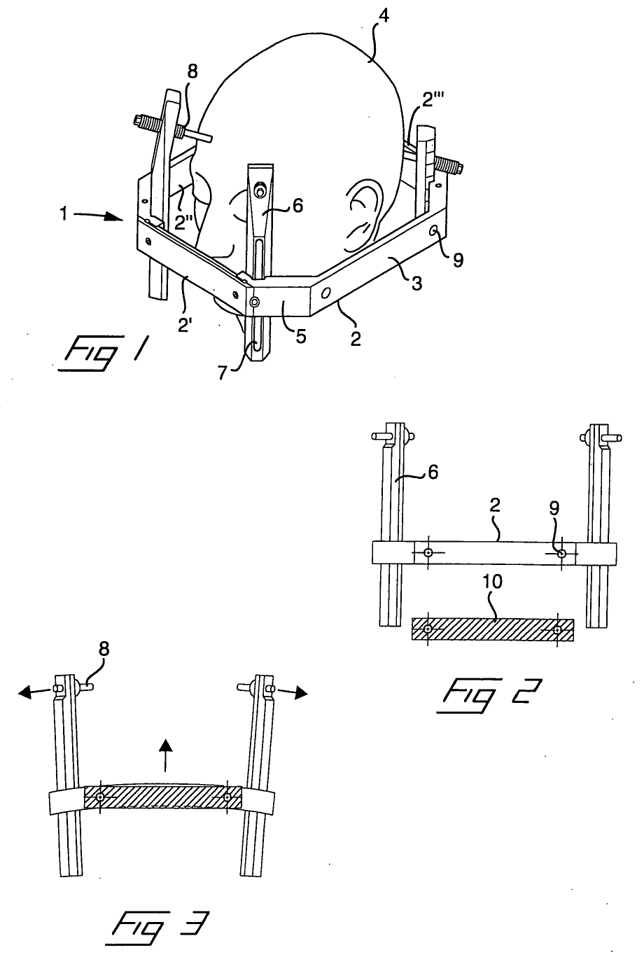

Reference is first made to FIG. 1, in which an embodiment of a device according to the invention is shown in a perspective view. The device comprises a frame, generally indicated by reference number 1, which is assembled of four individual frame members 2, 2′, 2″, 2′″ into a general rectangular, ring-formed shape. Each frame member includes a straight and flat portion 3 and the two frame members 2, 2″, which are adapted to be located at each side of a head 4, also includes a corner portion 5 in each end.

The device also comprises, preferably but not necessary, four pin support members 6, each of which is located in a respective corner portion and adjustable mounted by way of a slot 7 through the pin support member. In the upper end of each pin support member, a fixation pin 8 is inserted through a corresponding hole in the pin support member, such that a pointed end of each fixation pin may be threaded into abutment against the skull and tightened for fixation of the device thereto. The device is also provided with connecting means, which in this embodiment are depicted as plain holes 9 through each of the side frame members 2, 2″.

In FIGS. 2 and 3, the device according to the invention is shown in side elevations together with a guide member 10. The guide member may, preferably but not necessarily, be provided with a measuring scale to facilitate setting of the equipment in the proper position. In FIG. 2 the device is in an unloaded state and the guide member still not mounted. When loading the device, by threading the fixation pins 8 to abutment against the skull (not shown) and tightening of the fixation pins for rigid fixation to the skull, the device will be deformed, as is depicted in an exaggerated way in FIG. 3, such that the upper ends of the pin support members 6 will be deflected outwards and the frame member will be bent upwards, as is illustrate by the arrows. By placing the connecting means 9 in positions where the frame has a minimum of displacement due to the deformation, in accordance with the invention, the guide member 10 may easily be mounted onto the frame member 2 without the need for providing any extra play in the connecting means at the frame and/or the guide member. Neither will the guide member bend or deform in any other way as long as each connection is at least limited rotatable. This can be accomplished in any arbitrary way, e.g. by means of a screw joint.

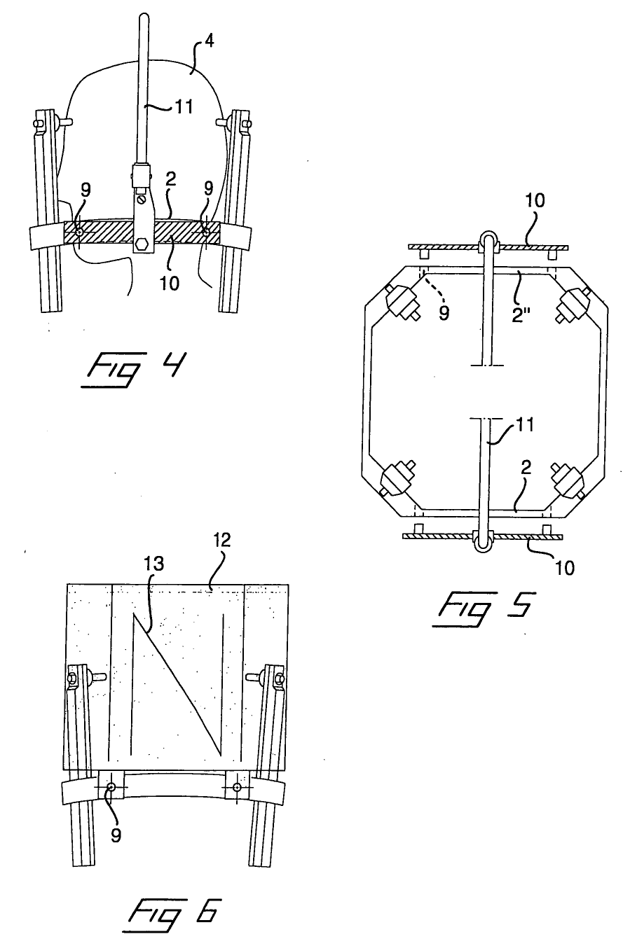

One single guide member 10 may be used for slidingly support any arbitrary equipment on the device. However, it is also possible to provide two parallel guide bars on opposite side frame members 2, 2″, for sliding supporting of a holding fixture 11, as is shown in FIGS. 4 and 5, which in its turn can be used for supporting additional equipment such as a surgical instrument or needle for treatment or taking of samples inside the head.

In FIG. 6, the device according to the invention is shown supporting an indication box 12 of transparent plastic having N-shaped fiducials or indication markings 13, which are visible in a chosen imaging process. By connecting the indication box to the frame at the connecting means 9, it is no need for any mounting play between the box and the frame due to the deformation of the frame. Accordingly, the precision of the imaging process will be increased.

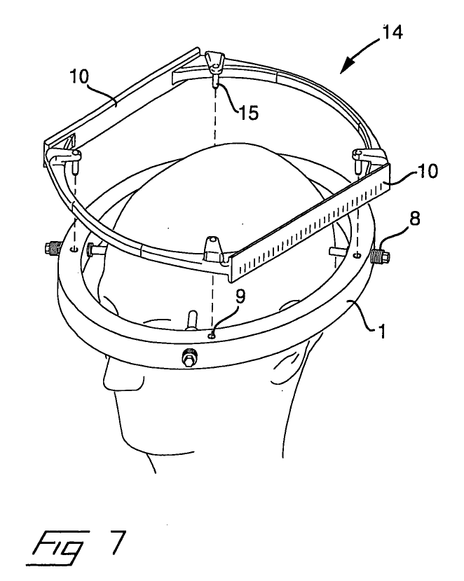

In FIG. 7 is shown an alternative embodiment of a frame 1 according to the invention. This frame is elliptic and preferably made of a synthetic material to be inert in relation to the imaging method used. Therefore it can be positioned at the level of the patient's forehead without distorting the imaging process. Accordingly it is also attached to the head by means of fixation pins 8, which are threaded directly through the frame without any need for pin support members as in the previous embodiment. In the figure is also shown an equipment in form of two guide bars 10, which are provided with measuring scales and intended for displaceable mounting of not shown additional equipment, such as a holding fixture for surgical instruments or needles. However, in this embodiment the guide bars are interconnected such that the guide bars in their turn form a guide frame 14 which is supported on the upper side of the frame 1. Accordingly, also the connecting means, in form of holes 9, are positioned in the upper side of the frame in locations where the frame 1 has a minimum of displacement due to the deformation of the frame when tightening the fixation pins against the head. The guide frame 14 has corresponding pins 15 for fitting into the holes 9 in the frame.

1. Device for fixation of equipment to the head (4) of a patient during neurological diagnosis, therapy or surgery, comprising a ring-shaped frame (1) having fixation pins (8) which are attachable against the skull of the patient with a contact force for fixation of the device thereto, wherein at least one connecting means (9) is positioned on the frame at a location where the frame (1) has a minimum of displacement due to deformation of the frame when the fixation pins (8) are tightened towards the skull.

2. Device according to claim 1, wherein the equipment (10, 11, 12) for diagnosis, therapy or surgery comprises at least one straight guide member (10), which is attachable to one of the sides of the frame (1) at two connecting means (9) for forming of connections which are at least limited rotatable, wherein additional equipment (11) for diagnosis, therapy or surgery is sliding attachable to the guide members.

3. Device according to claim 1, wherein the equipment for diagnosis, therapy or surgery comprises a device provided with fiducials (13), which are visible in any employed image producing method for diagnosis, and which, when the device is attached to the frame (1) at the connecting means (9), has a known position in relation to the frame.

4. Method for fixation of equipment to the head (4) of a patient during neurological diagnosis, therapy or surgery, comprising the steps of
providing a frame (1) having a ring-shaped configuration,
connecting fixation pins (8) to the frame and tightening the fixation pins against the skull of the patient with a contact force for fixation of the frame thereto,
attaching additional equipment (10, 11, 12) for diagnosis, therapy or surgery to the frame in at least one position where the frame has a minimum of displacement due to deformation when tightening of the fixation pins against the skull.

5. Method according to claim 4, comprising the further step of attaching the equipment (10, 11, 12) to two connecting means (9) for forming of connections which are at least limited rotatable.
